(19) United States
(12) Patent Application Publication
(10) Pub. No.: US 2012/0251639 A1
(43) Pub. Date: Oct. 4, 2012

(54) Mole Eliminating Ointment

(76) Inventor: Mark Smith, Pagedale, MO (US)

(21) Appl. No.: 13/079,133

(22) Filed: Apr. 4, 2011

(51) Int. Cl. A61K 36/752; A61P 17/00; A61K 36/81

(52) U.S. Cl. 424/736

(57) ABSTRACT

A topical treatment, and a method for making and using, for eliminating moles comprising, a mixture of natural extracts, where said extracts include garlic, white potatoes, onions and grapefruit.

### BACKGROUND OF THE INVENTION

1. Field of Invention

The present invention relates to a topical herbal treatment for moles that provides a safe means for removal.

2. Description of Related Art

Topical treatments include creams, gels, lotions or ointments that may be applied to the body surface, in particular the skin or particular membranes such as the nose, ear or throat, for treatment of particular conditions on the surface of the skin. Many topical ointments or lotions that are applied to the skin include medical formulations that have been developed to treat the particular condition. Topical treatments have also been known to be in the form of droplets from a small container. Further some topical treatments may be used in conjunction with a patch, sponge, tape or other application means to place the ointment, cream, lotion or other formula on the skin surface. Some topical treatments treat specific disorders on the skin such as sunburn, rashes, skin tone and in some cases moles that develop on the skin. Other treatments have been developed for treatment of warts that may also develop on the skin. Many times an individual may wish to treat a mole or wart that may appear on the skin surface for cosmetic purposes. Others wish to treat these skin disorders to avoid abrasions that may be caused due to the positioning of the mole or wart such as moles that may appear along the neckline or wrist. Many of the topical treatments that are presently available for the treatment of moles are chemically based with solvents or involve expensive medications that are sold through prescriptions. Consequently, there is always a need for a homeopathic remedy for safely removing moles from the surface of the skin. Use of such type of remedies may help to alleviate the necessity for a chemically based or expensive prescription ointment that may be in the prior art.

### SUMMARY OF THE INVENTION

The present invention relates to a topical treatment, and a method for making and using, for eliminating moles comprising, a mixture of natural extracts, where said extracts include garlic, white potatoes, onions and grapefruit.

### DETAILED DESCRIPTION

The present invention relates to a topical herbal homeopathic ointment for the treatment of moles. The treatment in accordance with the present invention is applied directly on to the skin and it includes a mixture of natural ingredients that causes moles to peel off the skin surface. The present invention includes a unique mixture of extracts from garlic, white potatoes, onions and grapefruit. This combination is applied directly to the skin through the use of a cotton swab. This mixture is developed through the use of a conventional juicer and based upon methods in accordance with the present invention the mixture creates a natural solvent that may effectively remove a mole.

The present invention may be developed as a mixture of garlic, white potato, onions and grapefruit extracts to form a liquid or cream topical skin treatment. Once the mixture is developed the treatment may be applied upon any mole on the surface of a user's skin. Application of this topical treatment effectively removes moles that may be harmful or bothersome to a user. Use of the topical treatment in accordance with the present invention effectively removes moles from the skin of a user and is safer than the use of any chemical or prescription based ointments presently on the market.

The mole eliminator is created with a mixture that includes garlic, onions, white potatoes and grapefruit as mentioned above. When creating the solution for application to the subject mole, these ingredients are sliced and placed into a conventional juicer. Once in the juicer, the contents are blended into a liquid composition. The liquid composition may then be applied to the surface of the skin where the mole may exist. Application of this liquid may be accomplished by the use of a cotton ball or other similar application device. The substance is applied on a daily basis until the mole starts peeling usually within a three to four week period. Normally after application, the user may feel a tingling or itching sensation that provides an indication that the solution is beginning to work on removal of the mole. Some individuals may experience some redness, soreness or other skin irritations that may be alleviated by the use of a Vaseline solution placed over the area. In an alternative embodiment, the mole treatment solution may be developed in the form of a cream ointment and therefore it alleviates the necessity for an applicator such as a cotton ball.

What is claimed is:

1. A topical treatment for eliminating moles comprising, a mixture of natural extracts, where said extracts include garlic, white potatoes, onions and grapefruit.

2. A method of creating a topical treatment comprising the steps of:
a. placing garlic, white potatoes, onions and grapefruit into a juicer; and
b. initiating the juicer to create a liquid substance.

3. The method according to claim 2, where the liquid substance is formulated into a cream or ointment.

4. A method for eliminating a skin surface mole comprising:
a. placing garlic, white potatoes, onions and grapefruit into a juicer;
b. initiating the juicer to create a liquid substance;
c. using an applicator to apply a portion of the liquid substance to a skin surface mole;
d. reapplying the liquid substance on a daily over a period of time; and
e. ceasing application of the liquid substance once the mole peels off the skin surface.

5. The method according to claim 4, wherein said period of time includes a range of three weeks to four weeks.

6. The method according to claim 4, where the liquid substance is formulated into a cream or ointment.
